(19) United States
(12) Patent Application Publication
(10) Pub. No.: US 2007/0237728 A1
(43) Pub. Date: Oct. 11, 2007

(54) Method and Composition for Dental Bleaching

(75) Inventor: Peter VERHEYEN, Meeuwen-Gruitrode (BE)

Correspondence Address: BROWDY AND NEIMARK, P.L.L.C.;624 NINTH STREET, NW, SUITE 300, WASHINGTON, DC, 20001-5303, US

(73) Assignee: HIGH TECH LASER, Meeuwen-Gruitrode (BE)

(21) Appl. No.: 11/763,918

(22) Filed: Jun. 15, 2007

Related U.S. Application Data
(63) Continuation of application No. 10/380,431, filed on Jul. 22, 2003, now abandoned.

(51) Int. Cl. A61K 6/00

(52) U.S. Cl. 424/53

(57) ABSTRACT

This invention relates to a method for bleaching teeth that have been discoloured by a staining agent. The method comprises the steps of coating an area of at least one tooth to be bleached with a dental bleaching composition and irradiating the coated area with laser energy for a predetermined period of time to activate an oxidising agent contained in the bleaching composition, the activated oxidising agent being capable of reacting with the staining agent to at least partly discolour the staining agent. In the method of this invention use is made of a laser emitting laser energy of a wave length capable of inducing a photochemical generation of radicals of the oxidising agent, which radicals in turn are capable of reacting with the staining agent to form a compound that is free of a conjugated electron system capable of absorbing visible light.

### CROSS REFERENCE TO RELATED APPLICATION

This is a continuation of U.S. application Ser. No. 10/380,431, filed on July 23, 2003, as a national stage of PCT BE 00/00107, filed on Sep. 14, 2000.

The present invention relates to a method for dental bleaching as described in the preamble of the first claim.

Over the centuries, an ongoing interest has existed in seeking solutions for whitening teeth. In essence, teeth discolouration can be subdivided in two main categories: extrinsic discolouration of the outer surface of the teeth and intrinsic stains located within the crystalline structure of the dental enamel and dentine.

Extrinsic discolouration or stain can be caused by a wide variety of substances including, coffee, tea, tobacco, fruit (for example berries), etc. Extrinsic stain can mostly be removed by dental instruments or polishing abrasives. However, when the degree of penetration of the stain into the tooth is too high, existing methods often fail. Intrinsic discolouration may originate from a wide variety of sources. It may be endogenous or exogenous or have taken place in the course of or after odontogenesis.

One of the main causes of intrinsic teeth discolouration is tetracycline staining. Tetracycline antibiotics were introduced in 1948 as broad spectrum antibiotics, for remedying respiratory illness and cystic fibrosis of children. Staining due to administration of tetracyclines has been reported for the first time in 1956. Staining by tetracyclines is attributed to the incorporation thereof into the hydroxyl apatite crystals which form the mineral dentine portion of the tooth. This incorporation mostly takes place in the course of the mineralisation stage which is part of the tooth development. Some accumulation of tetracyclines in the tooth enamel has also been observed, as well as hypoplasia of the tooth enamel. Discolouration may vary from yellow to yellow-brown, brown, grey or blue, sometimes accompanied by the formation of stripes on the tooth. Often bilateral discolouration is observed, covering both arches of the mouth. The degree of discolouration varies a.o. with the age at which the tetracyclines have been administered. It has been found that front milk teeth are sensitive to tetracyclines at an age of 4 months prior to the birth of the child up to an age of nine months of the baby. Final dentition appears to be sensitive from an age of 3 months up to approximately 7 years. The degree of discolouration has been found to vary further with the duration of administration, the tetracycline dosage applied and the type of tetracycline. Tetracycline chloride (auromycin) has been found to involve a grey-brown discolouration, dimethylchlorotetracyclin (ledermycin) a yellow discolouration, oxytetracyclin (terramycin) and tetracycline (achromycin) a yellow discolouration.

Another cause of teeth discolouration is fluorosis, which is mainly observed when an excess of fluoride is present during the formation of the enamel matrix and calcification. Fluorosis often involves hypoplasia of the enamel matrix and white spot lesions, which may even be accompanied by pronounced pitting of the enamel. It is believed that the discolouration is caused by extrinsic factors following dental eruption featuring roughness. White spot lesions may however also be the result of trauma or genetic malformations, fever, specific diseases. The main cause of white spot lesions is local demineralisation of the enamel. Discolouration of teeth may further be imparted by chromogenic bacteria, components present in saliva which may result in a brown colouration of the teeth, products originating from blood, phenyl ketonuria, erytroblastosis fetalis, sickle cell anaemia, amelogenesis and dentogenesis imperfecta.

External bleaching techniques wherein the discolouration agents are oxidised by an oxidising agent, mostly hydrogen peroxide, appeared to be unsatisfactory. The temporary character of this bleaching technique resides in the fact that the overlying enamel is relatively impermeable, thus limiting the bleaching effect to a depth of approximately 7 microns. As the majority of the tetracycline stain resides in the dentin, the result is a superficial bleaching of the teeth.

From WO98/23219 a method for dental bleaching is known in which the teeth to be bleached are coated with a topical dental bleaching composition, whereafter the coated teeth are irradiated with an argon laser. The dental bleaching composition comprises (1) hydrogen peroxide as a bleaching agent, (2) silicon dioxide as an inert gelling compound, (3) a discrete inert particulate material in a colour complementary to the colour of the laser used in conjunction with the composition, (4) a compound for adjusting the pH to about 9-11. The components (1)-(4) are mixed to form a translucent or transparent gel. The discrete colourant particles are capable of absorbing light emitted by the laser and of re-transmitting it as thermal energy to increase the reactivity and thus the effectiveness of the bleaching composition. After the coated tooth surfaces have been exposed to the laser energy the coating is removed, preferably by rinsing. The process can be repeated until the desired degree of bleaching is achieved.

The blue argon laser which has a wave length of 488 nm for 75% of the emitted energy and 514.5 nm for 25% of the emitted energy, absorption of the laser energy by the composition results in a heating thereof, which initiates the reaction responsible for the discolouration of the teeth. Due to the nature of the laser light used, the laser is only capable of involving a thermal effect in the composition. Preferably a laser with a power density of 350-550 milliwatts/cm² is used to avoid that the temperature of the teeth raises too high during the treatment and to reduce post bleaching sensitivity of the teeth. This goes however at the expense of effectiveness of the method.

It is the aim of the present invention to provide a dental bleaching method with which post bleaching sensitivity of the teeth can be further reduced.

This is achieved according to the present invention with the technical features of the characterising part of the first claim.

In the method of this invention use is made of a laser which emits energy of a wave length capable of inducing a photochemical reaction to form radicals of the oxidising agent capable of reacting with the staining agent.

The photochemical way of generating radicals reactive with the staining agent allows to minimise heating of the irradiated area of the tooth and thus to minimise post bleaching sensitivity of the teeth. As post bleaching sensitivity is minimised, the additional application of a fluoride compound to reduce this post bleaching sensitivity becomes superfluous and can be avoided.

Simultaneously with the minimised heating of the teeth following irradiation with laser energy, it has been found that the photochemical activation provides a higher intrinsic over-all radical yield than thermal activation, and that also the rate at which radicals are generated is higher as compared to thermal activation. As with the current method heating of the teeth is minimised, higher laser energies can be used, so that the over-all radical yield per time unit can be further increased. In the method of this invention, laser energy densities of between 1000 and 2600 mW/cm² can be used. With such energy densities the time needed for bleaching teeth can be decreased, thus leading to a method with an improved efficiency.

Due to the improved radical generation and the limited heating of the teeth, a dental bleaching method is provided which is fast and is comfortable to the patient.

In the method of this invention, preferably a laser is used which emits laser energy with a wave length of between approximately 525-545 nm, preferably of approximately 532 nm. An example of a laser capable of emitting 532 nm laser light is a KTP (potassium-titanyl-phosphate) laser, but other lasers emitting energy within this range can also be used.

The inventor is of the opinion that the unexpected effect of the 532 nm laser can be explained by a synergistic effect. Besides that fact that this laser energy appears to be capable of providing a high radical yield of the oxidising agent, it also appears to be capable of already inducing as such a decomposition reaction of the staining agent. The fact that the conjugated electron system of the staining agent which is responsible for the discolouration of the tooth, can already be at least partly decomposed upon irradiation with laser energy results in a first discoloration of the tooth. After this first breakdown has occurred, the at least partly decomposed staining agent molecules are capable of reacting with the oxidising agent radicals, thus inducing a further breakdown of the conjugated electron system. As the radicals of the oxidising agent can be built into the molecular structure of the decomposed staining agent, stabilised reaction products can be obtained, which are no longer reactive and are substantially free of conjugated π-electron systems capable of absorbing visible light. As these molecules do no longer contain a conjugated π-electron system capable of absorbing visible light, they will rather reflect visible light than absorbing it. Besides this, the use of a laser source emitting energy outside the UV wave length range allows to minimise the risk to damaging the tooth structures for example the enamel, the dentine and the dental nerve system and to minimise the risk to the occurrence of mutagenic or carcinogenic effects.

In the method of this invention, preferably use is made of a dental bleaching composition containing a compound capable of at least partly absorbing the light emitted by the laser. Thereto, the composition contains an absorbing agent with a colour substantially complementary to the laser light. As the colour complementary to green is red, preferably chromophores with a red or reddish colour are used. After the laser energy of the particular wave length has been absorbed by the red chromophore, it becomes available to the oxidising agent present in the bleaching composition to generate the above described radicals. The laser energy absorbing chromophores thus ensure that even in case the bleaching composition has been absorbed into the interior of the dentine of the tooth, the bleaching composition is still capable of absorbing the laser energy and of reacting with the staining agent. In that way the removal of the staining agents from the teeth is not limited to the surface thereof, but may proceed towards the interior of the tooth.

In the method of this invention, preferably use is made of a dental bleaching composition containing an oxidising agent suitable for use in dental applications. The oxidising agent is preferably selected from the group of peroxides, for example hydrogen peroxide, carbamine peroxide; perborates, for example sodium perborate; oxalic acid when treating iron stains. Hydrogen peroxide is the preferred oxidising agent as it is capable of generating the reactive perhydroxyl radical, which can be built in the molecular structure of the staining agent as a hydroxyl group. In that way a stabilised decomposition product of the staining agent can be obtained, which is no longer capable of absorbing but mainly reflects light in the visible region.

The concentration of the hydrogen peroxide may vary from approximately 30-80 wt. %, preferably from approximately 35-50 wt. %, although 38-42 wt. % is preferred since in this concentration range the ultimate bleaching efficiency can be obtained while simultaneously minimising the risk to the occurrence of structural changes in the dentine or the enamel.

The dental bleaching composition used in the method of this invention preferably has a pH of between 8.5-10.5, preferably of between 9-9.5. Use of a composition with a lower or a higher pH results in a less effective discolouration of the staining agent. This is probably due to the fact that outside of the preferred pH range, preferentially oxygen radicals are generated, which appear to show a lower activity.

This pH range of 8.5-10.5, preferably 9-9.5 is preferred as it involves the preferential generation of perhydroxyl radicals over other less reactive radicals generated at other pH-ranges. It has surprisingly been found that perhydroxyl radicals are capable not only of removing yellow and brownish discolouration, but also of removing grey discolouration which up to now could hardly be removed or could only be removed using extensive treatments involving serious after treatment pains. As the number of times the composition must be applied to the teeth to obtain a satisfactory discoloration can be limited, demineralisation of the teeth an irreversible damage of the dental nerve can be avoided. Demineralisation of a tooth and irreversible damage of the dental nerve is often found when the teeth are coated with H₂O₂ based compositions with an acid pH, that need to stay 30 minutes on the tooth to obtain some discoloration of the tooth. The bleaching methods based on the action of H₂O₂ as such are mostly based on the removal of a few mm of enamel from the tooth. As the thickness of the enamel layer of the tooth varies from 0.5-0.6 mm on the top of the front teeth to a maximum thickness of approximately 1.5 mm on the plane of some of the teeth, it can easily be understood that a repeated H₂O₂ may involve serious damage to the teeth.

In the method of this invention the area of the teeth to be bleached is cleaned, cleared of any debris and preferably also moisture is removed from the area to be treated as much as possible, the area to be treated is dried as good as possible before applying the bleaching composition. The cleaning of the teeth and removal of any debris can for example be done by means of a combination of pressurised air, water and sodium bicarbonate, or by applying a mixture of pumice and water. This cleaning step is important as enzymes present in the saliva may contain enzymes capable of decomposing hydrogen peroxide according to the following reaction:
2 H₂O₂→H₂O+O₂
thus reducing the amount of peroxide available for discolouration of the staining agent. Use of conventional polishing pastes is preferably avoided as they may contain oils which inhibit the action of the bleaching composition.

The drying of the area to be bleached is done to counteract interaction of the moisture with the bleaching composition, in particular the H₂O₂ present therein, before it has been capable of interacting with the staining compounds. Besides this, it has been found that penetration of the hydrogen peroxide radicals can be improved if the tooth surface is dried. The inventor is of the opinion that this must be attributed to the fact that the enamel and/or the dentine of the teeth is usually associated with a hydration shell, giving rise to a double layer with a high zeta-potential. The thus created strong electrical field by which the ionic permeability of the enamel and/or dentine is increased. As the accessibility of the double layer is improved, the permeability of the enamel and/or dentine can be increased.

The dental bleaching composition used in the method of this invention further contains a thickening agent in an amount of 80-98 wt. % with respect to the total weight of the composition, preferably in an amount of 90-98 wt. %. An amount of 94-95 wt. % is ultimately preferred as in this weight range an optimum gellation of the mixture can be obtained, thus improving the applicability of the bleaching composition.

Suitable thickening agents include silica, aluminium oxide, mixed silica-aluminium oxides, sodium stearate, long chain hydrocarbons for example Carbopol®, Trolamine®, Polyox®. Preferably fumed silica is used as it provides the most efficient distribution/reflection of the laser energy within the composition and allows the laser energy to penetrate within the dentine and involve photo bleaching of the dentine.

The present invention also relates to a dental bleaching composition containing a bleaching agent capable of interacting with a staining agent present in teeth, a pH adjusting compound, a thickening agent and a laser absorption enhancing compound, which is characterised by the presence of a bleaching compound capable of forming radicals upon irradiation with 525-545 nm laser energy. The bleaching compound is preferably present in an amount of 30-80 wt. %, preferably 38-42 wt. % of the total weight of the bleaching composition.

Other preferred embodiments of the dental bleaching composition have been described earlier on, when discussing the method of this invention.

If so desired, the method of this invention can be completed by a tooth strengthening fluoride after treatment, although this is not necessary to avoid after treatment pain. The fluoride after treatment is mainly intended as a preventive treatment to provide stronger teeth, less sensitive to caries and not as a pain reducing treatment. Although the method of this invention provides a stabile result with which any re-staining is hardly observed, use of the maintenance gel is advised to remove any staining that might have occurred following use of coffee, tea, wine etc. In the method known from the art, it is usually required that the teeth are covered with a tray containing a bleaching gel to ensure that the bleaching obtained after the laser treatment is not adversely affected or to avoid that the bleaching effect disappears.

The after treatment or maintenance gel comprises an amount of a fluoride compound, preferably 0.5-5 parts by weight with respect to the total weight of the composition, and an amount of a peroxide compound, preferably 5-15 parts by weight, to ensure that new staining that might have occurred after the bleaching treatment took place, is immediately removed. The abrasive properties of this gel have been found to be negligible. Its use is recommended after the last intake of food has been done. It can be applied with a tooth brush and be used similar to a regular tooth paste. When used on an every day basis, for a period of two weeks, the result of the bleaching of the method of this invention can be further improved.

Suitable examples of chromophores for use in the dental bleaching composition and method of this invention, capable of absorbing the laser energy used in the method of this invention include various types of Rhodamine®, acid Fuchsin, Alizarin Red, Basic Fuchsin, Carmine, Congo Red, Darrow Red, Oil Red O, Methyl Orange, Natural Red, Orange, Methyl Red, Chlorphenol Red, Phenol Red. All these compounds are commercially available from Aldrich® Chemie. Also a black compound may be used as a black would absorb all wavelengths of emitted light. Preferably however use is made of Rhodamine B® as it has an absorption peak around 532 nm, which is the closes to the energy of the laser light used in the method of this invention. In that way an optimum absorption of the laser energy can be ensured. Rhodamine is preferably present in a concentration of 0.25-1 wt. % with respect to the total weight of the composition, more preferably 0.5-0.6 wt. %. Above this range no improvement could be observed, below this range often an unsatisfactory discoloration was found.

Particulate material made of a compound capable of absorbing the laser energy with the wave length used in the method of this invention, may also be used. Suitable examples of such a materials include those materials that are not reactive with hydrogen peroxide and that will not leach their colour into the tooth surface or tooth structure in the course of the bleaching process. Suitable materials include coloured or coated porcelain, ceramic, thermoplastic compounds or polymeric resins such as polyacryl, acrylic resins, cellulose based resins, ceramic fibre compounds, fluoroplastic resins, polyamide resins, polycarbonate, phenolic resins, polyethylene, polyester, polymethyl-pentene resins, polyoxymethylene resins, polyphenylene resins, polypropylene, polystyrene, polyvinyl compounds, nitrile resins, terephtalic resins, glass fiber compounds etc. However, other types of polymeric materials known to the man skilled in the art may also be used. The particles may be coated with the laser energy absorbing compound, or the compound may be distributed within the particles. Such materials will be usually prepared according to techniques well known to the man skilled in the art.

The mean particles size of the particles of the gelling or thickening agent and other particulate material may vary within wide ranges and preferably ranges from about 50 microns to about 400 microns, more preferably from about 75 microns to about 200 microns, and most preferably from about 90 microns to 125 microns.

Adjustment of the pH to the preferred pH region of 8.8-10.5 is achieved by addition of a compound or mixture of bases suitable for use in dental applications. Suitable examples of such bases include sodium carbonate, sodium hydroxide, sodium polysilicate, but other bases known to the man skilled in the art and suitable for oral application may also be used.

Suitable thickening agents include inorganic oxides, for example silica, aluminium oxide, mixed silica-aluminium oxides, sodium stearate, or long chain hydrocarbons for example Carbopol®, Trolamine®, Polyox®. Preferably fumed silica is used. Preferred thickening agents are those capable of reflecting and scattering the laser light within the composition. In that way it is ensured that even in a part of the composition that may have been absorbed into the tooth, radical formation and the therewith associated discolouration of the staining agent takes place.

Suitable peroxides for use in the after-treatment gel of this invention include carbamide peroxides, for example ureum peroxide, but other peroxides may also be used. Suitable fluoride compounds include sodium fluoride.

The invention is now further elucidated in the following examples.

### EXAMPLE 1

Application of the Bleaching Composition of this Invention.

A powder mixture was prepared by mixing 400 mg of colloidal silica with a mean particle size of approximately 200 μm (Federa, Belgium) with 2.4 mg of Rhodamine B, and 20 mg of sodium carbonate to obtain a pH of approximately 9.5. To the powder mixture, approximately 5 ml of H₂O₂ 55 vol. %, alkaline solution was added, following which a gel was obtained of which the H₂O₂ was decreased with 25% as compared to the amount added. The mixture was left in a closed vessel for approximately 5 minutes. The given amounts appeared to be sufficient for bleaching of a complete dentition.

The teeth that needed bleaching were thoroughly cleaned, freed of any debris by treating them with a flow of pressured air, sodium bicarbonate and water. In another application this was done using a mixture of pumice and water.

A cheek retractor is inserted in the patient's mouth, to spread the lips and cheeks. Then, a cotton mouth dry field system is applied and connected to a water aspiration system to maintain the dental arches at a fixed distance from each other and to avoid contact of the bleaching composition with the tongue in the course of the treatment. An intensive profylaxis was done to remove all plaque and debris from the teeth in order to allow that an optimal bleaching is obtained. To avoid contact of the bleaching gel with the gums, the gingival margins and sulcus were covered with a blocking gel. The blocking gel was a commercially available gel for example a gel containing a polymerisable monomer, for example dimethylacrylate monomers, which polymerises upon irradiation with visible light or with certain lasers emitting laser energy in the right range. The teeth were partly covered 1 mm cervical with the blocking gel, as well as exposed dentine and those teeth and the parts of the teeth that needed no bleaching. Care was taken that parts of the teeth to be bleached were not accidentally coated with the blocking composition as this would inhibit the action of the bleaching gel.

After cleaning, the teeth were coated with an amount of the bleaching composition with a pencil as follows: 11-21, 12-22, 13-23, 14-24, 15-25, followed by 41-31, 42-32, 43-33, 44-34, 45-35. Every tooth was irradiated with 0.6 Watt of a KTP laser of 532 nm for 30 seconds in the order given above. The laser energy was decreased in case the patient suffered any pain. After the gel had been in contact with the teeth for 10 minutes, it was sucked away, the teeth were rinsed with water and dried.

The bleaching gel was applied a second time in the following order: 21-11, 22-12, 23-13, 24-14, 25-15, followed by 31-41, 32-42, 33-43, 34-44, 35-45, and irradiated with the laser light. The desired bleaching effect was obtained.

After the treatment had been completed, the bleaching gel was removed and the teeth were rinsed with a copious amount of water. The teeth that had been exposed to the bleaching composition were covered with a fluoride gel to reinforce the teeth. The fluoride gel contained 3 wt. % of sodiumfluoride, 0.7 wt. % of citric acid, 1 wt. % of sodium monophosphate, 3 wt. % of hydroxyethylcelluloses, and water.

As can be understood from example 1, it is adviseable to start coating the teeth with the front teeth and to proceed from the front to the back teeth. The front teeth appear to have the thickes enamel layer, the thickness of the enamel layer decreasing from the front to the back of the mouth.

### EXAMPLE 2

Example 1 was repeated, this time making use of handpiece laser with a collimated beam. The power setting was 0.4 Watt in stead of 0.6 Watt. The desired bleaching effect was obtained.

### EXAMPLE 3

Example 1 was repeated. As the teeth had been intensively stained, the bleaching effect obtained after the treatment of example 1 was unsatisfactory.

The teeth were coated with the bleaching composition for a third time, this time starting as follows: 11-21, 12-22, 13-23, 14-24, 15-25, followed by 41-31, 42-32, 43-33, 44-34, 45-35. The teeth were irradiated with the bare fibre, 0.6 Watt of a KTP laser of 532 nm for 30 seconds in the same order as the bleaching gel was applied.

After rinsing with water and drying, a fourth application of the bleaching gel was done in the following order: 21-11, 22-12, 23-13, 24-14, 25-15, followed by 31-41, 32-42, 33-43, 34-44, 35-45.

Care was taken that a total irradiation time of 30 minutes was not exceeded to avoid that the tooth structure is adversely affected.

After the treatment had been completed, the bleaching gel was removed with water. The teeth that had been exposed to the bleaching composition were covered with a fluoride gel to reinforce the teeth. The fluoride gel contained 3 wt. % of sodiumfluoride, 0.7 wt. % of citric acid, 1 wt. % of sodium monophosphate, 3 wt. % of hydroxyethylcelluloses, and water.

### EXAMPLE 4

Maintenance Gel.

In the method of this invention a gel containing the following compounds was applied once and twice a week, before going to sleep: ureum peroxide 10 parts by weight, sodium fluoride 1 part by weight, carbopol 2 parts by weight, triethanolamine 4 parts by weight, citric acid 1 part by weight, sodium EDTA 0.1 parts by weight, sodium saccharide 0.2 parts by weight, mint flavour 0.2 parts by weight, sodium benzoate 0.1 parts by weight, water until 100 parts by weight.

Preferably approximately 1 ml of the gel is applied to a wet tooth brush, and divided over the upper and lower teeth both on the tooth surface facing the cheeks and the inner tooth surface and scrubbed for approximately 5 minutes. Care was be taken not to swallow the gel. The gel was spit out, no rinsing was done.

The inventor believes that the effect of the dental bleaching method and the bleaching composition of this invention can be explained as follows. Upon irradiation with laser energy, part of the laser energy contacting the bleaching composition, is absorbed by the absorbing agent present in the composition and transferred to the teeth. The absorption of the laser energy becomes clear from the discoloration of the bleaching composition after it has been irradiated with laser energy. Part of the laser energy impinging the composition is reflected within the composition, until finally at least part of it contacts the teeth and is absorbed by the staining agents. As irradiation proceeds, at least part of the absorbed and reflected laser energy penetrates the enamel and the dentine, the penetration depth increasing with time. The penetration of the laser energy into the tooth structure ensures that the discoloration is not limited to the surface of the tooth, but rather proceeds into the tooth structure, thus providing an in depth discoloration of the tooth.

The method and composition of this invention are suitable for bleaching integrally intact teeth. In the case of repaired teeth, for example teeth containing a filling or a crown or when a bridge or prothesis is present in the mouth, it is preferably to adapt the method to the nature of the reparation. For example, after the original part of a repaired tooth has been bleached, the filling is removed and the thus created hole is re-filled with a composition the colour of which corresponds to the actual colour of the bleached teeth.

The method and composition of this invention are suitable for bleaching a complete set of teeth, a selected number of teeth, a single tooth or a-vital bleaching of one or more teeth. Care must be taken to avoid external resorption of the root, by avoiding that H₂O₂ to disseminate by the dentine tubulus of the root dentine into the periodontal ligament. Dissimination of H₂O₂ may namely give rise to a strange-body reaction and osteclastic activity. Care must be taken to carefully isolate the pulp cavity. The bleaching composition is preferably applied to the vestibular area and in the pulp cavity of the tooth. Each of them is then irradiated with laser energy for 30 seconds. After both the pulp and the vestibular area have been irradiated, it is adviseable to wait for an overall period of time of approximately 10 minutes at least, in the mean time repeating laser application on the buccal side and the lingual/palatal side of the tooth for 2 or 3 more times. The procedure is repeated the required number of times, preferably 3 to 4 times to obtain the desired effect. Preferably the procedure is terminated when the colour of the treated tooth is still somewhat darker than the colour of the adjacent teeth, as some discoloration will continue even after the composition has been removed from the teeth. This can most probably be explained by the fact that part of the composition may be absorbed into the tooth structure. As absorption of H₂O₂ or penetration of its radicals into the tooth is diffusion controlled, the reaction with the staining agent will most probably also be diffusion controlled.

1. In a method for dental bleaching, comprising coating teeth with a composition containing an oxidising agent capable of interacting with a staining agent present in teeth, a pH adjusting compound, a thickening agent and a laser absorption enhancing compound, the oxidising agent being a compound capable of generating radicals upon irradiation by the laser and irradiating the composition coated teeth with a laser the improvement wherein
the composition is irradiated with a laser emitting electromagnetic irradiation having a wavelength of approximately 525-545 nm so as to induce a photochemical reaction to the oxidising agent to form radicals reactive with the staining agent and to form a compound that is free of a conjugated electron system capable of absorbing visible light, whereby heating of the irradiated area of the tooth and thus post bleaching sensitivity of the teeth are minimized.

2. The method for dental bleaching as claimed in claim 1, wherein a laser is used emitting laser energy with a wave length of approximately 532 nm.

3. The method for dental bleaching as claimed in claim 1, wherein said compound has a color substantially complementary to the laser light.

4. The method for dental bleaching as claimed in claim 1, wherein said thickening agent has a mean particle size from about 50 micron to 400 micron.

5. The method of claim 4 wherein said particle size is about 75 micron to about 200 micron.

6. The method of claim 4 wherein said particle size is about 90 micron to about 125 micron.

7. The method for dental bleaching as claimed in claim 1, wherein said oxidising agent is selected from the group of consisting of at least one peroxides, perborates and oxalic acid.

8. The method for dental bleaching as claimed in claim 1, wherein said compound contains rhodamine as an absorbing agent.

9. The method as claimed in claim 8, wherein said rhodamine is present in a concentration of 0.25-1% by weight with respect to the total weight of the composition.

10. The method for dental bleaching as claimed in claim 1, wherein said oxidizing agent is a peroxide compound present in an amount of 30-80 wt %.

11. The method of claim 10 wherein said amount is 35-50 wt %.

12. The method of claim 10 wherein said amount is 38-42 wt %.

13. The method for dental bleaching as claimed in claim 1, wherein said composition has a pH of 8.5-10.5.

14. The method of claim 13 wherein said pH is 9-9.5.

15. A topical dental bleaching composition containing an oxidising agent capable of interacting with a staining agent present in teeth, a pH adjusting compound, a thickening agent and a laser absorption enhancing compound, wherein the oxidising agent is a compound capable of generating radicals upon irradiation with electromagnetic irradiation with a wave length of approximately 525-545 nm through photochemical reaction, the oxidising agent radicals being capable of reacting with the staining agent to form a compound that is free of a conjugated electron system capable of absorbing visible light, whereby heating of the irradiated area of the tooth and thus post bleaching sensitivity of the teeth are minimized.

16. A topical dental bleaching composition as claimed in claim 15, wherein the composition contains 30-50 wt. % of an oxidising agent.

17. The composition of claim 16 wherein said oxidizing agent is present in an amount of 38-42 wt %.

18. A topical dental bleaching composition as claimed in claim 15, wherein the laser absorption enhancing compound contains an absorbing agent capable of absorbing energy with a wave length of 525-545 nm.

19. A topical dental bleaching composition as claimed in claim 15, wherein the laser absorption enhancing compound contains a colorant selected from the group consisting of at least one of Rhodamine, acid Fuchsin, Alizarin Red, Basic Fuchsin, Carmine, Congo Red, Darrow Red, Oil Red O, Methyl Orange, Natural Red, Orange, Methyl Red, Chlorphenol Red, and Phenol Red.

20. A topical dental bleaching composition as claimed in claim 15, wherein the composition has a pH of 8.5-10.5.

21. The composition of claim 20, wherein said pH is 9-9.5.

22. The method of claim 1, wherein said compound has a red color.

23. The composition of claim 15, wherein said laser absorption enhancing compound has a red color.
